(19) United States
(12) Patent Application Publication
(10) Pub. No.: US 2006/0025378 A1
(43) Pub. Date: Feb. 2, 2006

(54) Dose dependent elimination of HA and cell receptor stimulation

(76) Inventors: Linda M. Pilarski, Spring Lake (CA); Eva A. Turley, London (CA)

Correspondence Address: GOWLING LAFLEUR HENDERSON LLP, SUITE 1400, 700 2ND ST. SW, CALGARY, AB, T2P 4V5, CA

(21) Appl. No.: 10/927,774

(22) Filed: Aug. 30, 2004

(30) Foreign Application Priority Data
Jul. 27, 2004 (CA) 2,475,762

(51) Int. Cl. A61K 31/728

(52) U.S. Cl. 514/54

(57) ABSTRACT

The invention provides a method to alter elimination kinetics of hyaluronic acid (HA) in a patient is disclosed. Administration of HA to a patient, followed by at least one subsequent administration results in an alteration of the ability of the patient to eliminate HA. Also disclosed is a method for upregulating, and/or increasing the affinity of cellular HA receptors. Also disclosed are methods for increasing the ability of cells to bind HA improved imaging of cells and tissues, determination of the existence of disease in a patient.

### FIELD OF THE INVENTION

The invention pertains to methods of administration of hyaluronic acid or pharmaceutically acceptable salts thereof (HA), in mammals, whereby the elimination rate of HA may be controlled and/or modulated. The invention further pertains to methods of altering receptor affinity or prevalence in specific cell types through administration of HA; and methods for disease detection in a mammal

### BACKGROUND OF THE INVENTION

HA is an ubiquitous tissue glycosaminoglycan (GAG) produced by three distinct HA synthases and degraded by five known hyaluronidases (McDonald, J. A. et al Glycoconj J 19:331 (2002); Spicer, A. P. et al Glycoconj J 19:341 (2002); Tammi, M. I. et al J Biol Chem 277:4581 (2002)). HA is retained in tissues by specific interactions with extracellular and cellular HA-binding proteins termed hyaladherins (Toole, B. P. Glycobiol 12:37R (2002)). Increased HA accumulation in tissues occurs during specific events in morphogenesis, wound repair, chronic inflammatory disorders, and upon neoplastic transformation (Toole, B. P. et al J Biol Chem 277:4593 (2002)). Enhanced accumulation of this GAG results from an altered balance in the activity and expression of synthases versus hyaluronidases (McDonald J. A. et al Glycoconj J 19:331 (2002)). Although the functions of enhanced HA accumulation in these physiological and pathological processes is only beginning to be dissected, HA is known to modify the physico-chemical nature of extracellular matrix and to contribute to the regulation of cell motility, invasion and proliferation (McDonald, J. A. et al Glycoconj J 19:331 (2002); Toole, B. P. Glycobiol 12:37R (2002)). The latter effects result from interactions of HA with cell receptors such as CD44 and RHAMM that activate intracellular signaling cascades to regulate these cell functions (Turley, E. A. et al J Biol Chem, 277:4589 (2002); Gares, S L. et al Dev Immunol 7:209 (2000)).

The ability of HA to activate signaling cascades appears to depend upon the molecular weight of HA, such that HA fragments are generally more able to activate signaling cascades than high molecular weight form (Tammi. M. I. et al J Biol Chem 277:4581 (2002); Toole, B. P. Glycobiol 12:37R (2002)) notably in vascular endothelial cells (Lokeshwar, V. B. et al J Biol Chem, 275:27641 (2000)). However, a number of studies suggest that specific sizes of HA oligosaccharides can also inhibit signaling (Toole, B. P. Glycobiol 12:37R (2002)).

HA is also present in plasma and reaches the vasculature from the tissues where it is synthesized via the lymph. Plasma levels can vary daily and have been reported to increase following injuries including liver fibrosis, arthritis, sepsis, thermal injury or during the progression of tumors such as melanoma, prostate cancer and malignant mesothelioma. The clearance of HA from plasma is largely due to uptake by endocytic HA receptors in endothelial cells in the liver, and possibly other tissues (Weigel. P. H. et al Biochim Biophys Acta 1572:341 (2002)). These cells can degrade HA completely or into smaller fragments (<25,000 daltons) that are released and ultimately excreted by the kidney (Weigel, P. H. et al Biochim Biophys Acta 15&2:341 (2002)). Elimination kinetics of serum HA present in a normal physiological range are linear and short, as assessed with rabbit and sheep animal models (Lebel, L. et al Pharm Res 6:677 (1989)). In these animal models, injection of HA (1.8 μg/ml) at concentrations higher than are normally present in plasma, markedly prolongs the half-life (4-10 fold) of serum HA. Elimination rates at these higher concentrations can best be described with non-linear or Michaelis-Menten kinetic modeling (Berg, S. J Intern Med 242:73 (1997)).

The kinetics of HA elimination in humans has also been reported for HA injected intravenously (IV) as either an infusion (Lebel L. et al Eur J Clin Invest 24:621 (1994)) or a single bolus (Torsteinsdottir, I. T. et al Semin Arthritis Rheum 28:268 (1999); Lindqvist, U. et al Clin Chim Acta 210:119 (1992)). In both paradigms the rates of HA elimination are non-linear and were modeled by Michaelis-Menten kinetics, (Lindqvist, U. T. et al Scand J Clin Lab Invest 57:49 (1997)). An average Michaelis-Menten constant (Kₘ) of 0.340 μg/ml and an elimination rate (Vₘₐₓ) of approximately 89-185 μg/min were reported in healthy volunteers (Torsteinsdottir, I. T. et al Semin Arthritis Rheum 28:268 (1999)).

Serum HA concentrations are reproducibly higher in patients with liver fibrosis or osteoarthritis and several studies have assessed whether or not differences in elimination kinetics contribute to the increase. In rheumatoid arthritis patients, the Vₘₐₓ constants for elimination of a bolus of HA in “loading” style experiments were higher than for healthy controls and reached significance in one study (Lindqvist, U. et al Clin Chim Acta 210:119 (1992)) but not in a subsequent assessment (Torsteinsdottir, I. T. et al Semin Arthritis Rheum 28: 268 (1999)). These studies suggest that a combination of measuring HA concentration in serum and an estimation of the Vₘₐₓ obtained after injection of a loading dose of exogenous HA may permit discrimination between healthy persons and persons with joint disease and be a useful marker with which to follow the course of this disease clinically. These methods suffer from inaccuracy and variation inherent between individuals; and though theoretically possible, are limited in practical application.

HA is possibly one of the most clinically utilized polysaccharides and has been administered in topical applications, for example for thermal wounds; as intrarticular injections, for example, for treatment of osteoarthritis in knee joint; as intra-occular injections for eye surgery; and following surgery for prevention of post-surgical adhesions. In addition to utilizing the remarkable visco-elastic properties of HA as a device in the above clinical settings, an increasing number of experimental studies have reported the use of HA for delivering drugs, for enhancing their efficacy or as a biomodifier. For example, HA has been used to coat injured tissues such as blood vessels to reduce restenosis and when administered as a specific sized fragment to induce tumor apoptosis and reduce tumor metastasis. Since HA is slowly released from its introduction site into blood and lymph, for example following intra-articular injection; and since it is clear that HA can be bioactive under specific conditions, it is important to understand the pharmacokinetics. More so, it is important to understand and control the sustained residency in mammals of injected HA.

In addition, both sonographic and MRI imaging is affected by the enhanced accumulation of hydrophilic polyanions such as HA. For example, increased HA accumulation at the edges of invading breast tumors contributes to the ability of ultrasound to detect the margins of the invading tumor (Vignal, P. et al J Ultrasound Med 21:532 (2002)). Further, increased HA associated with high grade gliomas results in an altered Apparent Diffusion Coefficient (ADC) as detected by MRI and this parameter can be used to differentiate between high and low grade glial tumors (Sadeghi, N. et al Am J Roentgenol 181:235 (2003)). Therefore, the ability to control accumulation and concentration of exogenously administered HA is of benefit.

Clinical HA products are applied at pharmacologically relevant doses and can result in prolonged elevation of HA plasma levels exceeding normal serum concentrations (Lindqvist, U. et al Am J Reontgenol 181:235 (2003)). The biological consequences of sustained pharmacological doses of HA will depend, in part, upon its rate of clearance, effects on endogenous HA receptor expression and the extent to which it is partially degraded into biologically active fragments by tissue or plasma hyaluronidases (Mio, K et al Maxtrix Biol 21:31 (2002)).

### SUMMARY OF THE INVENTION

It is therefore an object of the present invention to provide a method for altering the elimination kinetics of HA in vivo.

It is a further object of the present invention to provide a method for selectively increasing the ability of cells to bind HA in vivo.

It is a further object of the present invention to provide a method for detection of disease.

It is further object of the present invention to provide a method of selectively increasing the presence or affinity of cellular receptors capable of binding HA in vivo.

The present invention provides for a method to alter elimination kinetics of HA in a patient comprising administration of HA to the patient sufficient to result in a transient increase in blood HA presence followed by at least one subsequent administration of HA to the patient such that a transient increase in blood HA occurs.

In a preferred embodiment, the present invention provides for a method to alter elimination kinetics of HA in a patient wherein there is an administration of HA to the patient sufficient to result in a transient increase in blood HA presence followed by at least three subsequent administrations of HA to the patient each sufficient to result in a transient increase in blood HA presence.

In an alternate embodiment, the present invention provides for a method to alter elimination kinetics of HA in a patient wherein there is administration of four sequential dosages of HA to the patient of 1.5 mg/kg, 3 mg/kg, 6 mg/kg and 12 mg/kg.

In an alternate embodiment, the present invention provides for a method to alter elimination kinetics of HA in a patient wherein there is an administration of 4 sequential dosages of HA to a patient of 1.5 mg/kg, 3 mg/kg, 6 mg/kg and 12 mg/kg with a period of seven days between each subsequent HA administration.

In an alternate embodiment, the present invention provides for a method to alter elimination of kinetics of HA in a patient wherein the HA is administered by intravenous injection, oral administration, or subcutaneous injection.

Additionally, the present invention provides for a method to increase the ability of cells to bind HA comprising a multiplicity of HA administrations to the cell or cell environment.

In a preferred embodiment, the present invention provides for a method to increase the ability of cells to bind HA wherein the cells are in vivo.

In an alternate embodiment, the present invention provides for a method to increase the ability of cells in vivo to bind HA comprising an HA administration to a patient sufficient to result in a transient increase in blood HA presence; followed by at least one subsequent administration of HA to the patient sufficient to result in a transient increase in blood HA presence.

In an alternate embodiment, the present invention provides for a method to increase the ability of cells in vivo to bind HA comprising administration of HA to the patient sufficient to result in a transient increase in blood HA presence followed by three subsequent administrations of HA to the patient each sufficient to result in a transient increase in blood HA presence.

In an alternate embodiment, the present invention provides for a method to increase the ability of cells in vivo to bind HA comprising administration of four sequential dosages of HA to the patient of 1.5 mg/kg, 3 mg/kg, 6 mg/kg and 12 mg/kg.

In an alternate embodiment, the present invention provides for a method to increase the ability of cells in vivo to bind HA wherein there is a period of seven days between each of the subsequent HA administrations.

In an alternate embodiment, the present invention provides for a method to increase the ability of cells in vivo to bind HA wherein the HA is administered by intravenous injection, oral administration, or subcutaneous injection.

In an alternate embodiment, the present invention provides for a method to increase the ability of cells in vivo to bind HA wherein the cells are monocytes, macrophages, B-cells, CD4⁺ cells, CD8⁺ cells, white blood cells, chondrocytes, endothelial cells, fibroblast, breast endothelial or hematopoietic stem cells.

Additionally, the present invention provides for a method to increase the presence or affinity of cellular HA receptors comprising a multiplicity of HA administrations to the cell or cell environment.

In a preferred embodiment, the present invention provides for a method to increase the presence or affinity of cellular HA receptors wherein the cellular receptors are in vivo.

In an alternate embodiment, the present invention provides for a method to increase the presence or affinity of cellular HA receptors in vivo comprising administering a multiplicity of doses of HA to a patient, each sufficient to result in a transient increase in blood HA presence; followed by at least one subsequent administration of HA to the patient sufficient to result in a transient increase in blood HA presence.

In an alternate embodiment, the present invention provides for a method to increase the presence or affinity of cellular HA receptors in vivo comprising administration of HA to a patient sufficient to result in a transient increase in blood HA presence followed by at least three subsequent administrations of HA to the patient, each sufficient to result in a transient increase in blood HA presence.

In an alternate embodiment, the present invention provides for a method to increase the presence or affinity of cellular HA receptors in vivo comprising administration of four sequential dosages of HA to a patient of 1.5 mg/kg, 3 mg/kg, 6 mg/kg and 12 mg/kg.

In an alternate embodiment, the present invention provides for a method to increase the presence or affinity of cellular HA receptors in vivo wherein there is a period of seven days between each of the subsequent HA administrations.

In an alternate embodiment, the present invention provides for a method to increase the presence or affinity of cellular HA receptors in vivo wherein the HA is administered by intravenous injection, oral administration, or subcutaneous injection.

In an alternate embodiment, the present invention provides for a method to increase the presence or affinity of cellular HA receptors in vivo wherein the cells are monocytes, macrophages, B-cells, CD4⁺ cells, CD8⁺ cells, white blood cells, chondrocytes, endothelial cells, fibroblasts, breast endothelial cells or hematopoietic stem cells.

Additionally, the present invention provides for a method to detect disease in a patient comprising administration to a patient of a quantity of HA sufficient to result in a transient increase in blood HA presence, and at least one subsequent administration of HA to the patient sufficient to result in the transient increase in blood HA presence, followed by the identification and quantification of elimination kinetics of the HA in the patient; wherein alteration of the complexity of the elimination kinetics of HA in the patient is indicative of the presence of disease.

In a preferred embodiment, the present invention provides for a method to detect disease in a patient wherein alteration of the complexity of the elimination kinetics of HA in the patient is indicative of cancer, arthritis, or fibrosis.

Additionally, the present invention provides for a method to image cells or tissues of interest in a patient comprising administration of a quantity of HA to a patient sufficient to result in a transient increase in HA presence proximal to the cell or tissue of interest, at least one subsequent administration of HA to the patient sufficient to result in a transient increase in HA presence proximal to the tissue or cell of interest, administration of an imaging dose of HA and imaging of the cell or tissue of interest for the imaging dose.

In a preferred embodiment, the present invention provides for a method to image cells or tissues of interest in a patient wherein the imaging dose of HA comprises HA complexed with a radionuclide.

In an alternate embodiment, the present invention provides for a method to image cells or tissues of interest in a patient wherein the imaging dose of HA comprises HA complexed with ²⁴¹Am, ¹³⁷Ba, ⁴⁷Ca, ¹³⁷Cs, ⁵¹Cr, ⁵⁷Co, ⁶⁰Co, ⁶⁴Cu, ¹⁸F, ⁶⁷Ga, ¹⁹⁸Au, ¹²³I, ¹²⁵I, ¹³¹I, ¹⁹²Ir, ⁵⁵Fe, ⁵⁹Fe, ⁸⁵Kr, ²¹⁰Pb, ¹⁹⁷Hg, ²⁰³Hg, ³²P, ⁴²K, ²²⁶Ra, ¹⁰⁵Ru, ⁷⁵Se, ⁸⁵Sr, ^{87m}Sr, ²⁴Na, ³⁵S, ^{99m}Tc, ²⁰¹Tl, ³H, ¹³³Xe or ¹⁶⁹Yb.

In an alternate embodiment, the present invention provides for a method to image cells or tissues of interest in a patient wherein the imaging of the cell or tissue of interest is performed by magnetic resonance.

In an alternate embodiment, the present invention provides for a method to image cells or tissues of interest in a patient wherein the imaging of the cell or tissue of interest is performed by apparent diffusion coefficient magnetic resonance.

In an alternate embodiment, the present invention further provides for a method to image cells or tissues of interest in a patient wherein the imaging dose of HA contains a magnetic resonance imaging marker.

In an alternate embodiment, the present invention provides for a method to image cells or tissues of interest in a patient wherein the magnetic resonance imaging marker is ¹³C, ¹³P, ¹¹B, or ¹⁹F.

The accompanying drawings, which are incorporated in and constitute a part of this specification, illustrate preferred embodiments of the present invention and, together with the description, serve to explain the principles of the present invention.

### BRIEF DESCRIPTION OF THE FIGURES

FIG. 1 shows the quantification of serum HA levels following HA introduction of 1.5 mg/kg, 3 mg/kg, 6 mg/kg and 12 mg/kg administered as an IV injection infused over 2 hours. All values have been corrected for baseline, endogenous serum HA levels and are plotted as a linear dose versus time (hour) and values represent the mean of N=23.

FIG. 2 shows the values shown in FIG. 1 re-plotted as a log dose of HA versus time. Values represent the mean of N=23.

FIG. 3 shows the estimated slopes for each elimination curve. Values from FIG. 2 were forced through a linear line and the slopes were calculated for each dose.

FIG. 4 shows the binding of Fluorescein Conjugated HA (FITC-HA) to Peripheral Blood Monocyte cells (PBMC) (CD14⁺), B-lymphocytes (CD19⁺) and T-cells (CD4⁺ as well as CD8⁺) and was quantified using Fluorescence Activated cells sorting (FACS) analysis and sorting. Binding of FITC-HA to these cells types was quantified for all dosing periods using serum from 6 healthy volunteers following administration of HA of 1.5 mg/kg (FIG. 4A), 3.0 mg/kg (FIG. 4B), 6.0 mg/kg (FIG. 4C) and 12.0 mg/kg (FIG. 4D). Values represent the mean of N=6±S.E.M.

FIG. 5 shows the molecular weights of serum HA quantified by column chromatography following the final infusion of 12 mg/kg HA. The molecular weight of HA was determined 1 hour prior to the HA infusion, 0.5 hours after the beginning of the 2 hour HA infusion and 48 hours after the infusion.

### DETAILED DESCRIPTION OF THE PREFERRED EMBODIMENT OF THE INVENTION

The present invention demonstrates that HA infusions administered over a 4-week period are well tolerated by healthy humans, as no adverse events were observed that could be related to infusion of HA. Importantly, there was no evidence for a significant increase in HA fragmentation over this study period.

An understanding of the exact mechanisms by which exposure to multiple HA infusions increase HA binding to cells, in particular PBMC and B-cells; alter elimination kinetics in vivo; and increase cellular receptor presence of affinity for HA; is not required to practice the present invention. The mechanisms disclosed herein are intended to be non-limiting and serve only to better describe the present invention. A number of reports have shown that HA affects the expression of cell HA receptors such as CD44 and RHAMM (Savani, R. C. et al Int J Tissue React 17:141 (1995); Levesque, M. C. et al J Immunol 156:1557 (1996); Mytar, B. et al Int J Cancer 94:727 (2001)). Increasingly, reports note that many HA receptors exist in a constitutively inactive state with regards to their HA binding activity and that cytokines and phorbol esters can activate binding by post-translational modification of the receptor, for example CD44. Interestingly the expression of at least one of these cytokines, Tumor Necrosis Factor Alpha (TNFα), is increased in monocytes by HA, and HA may therefore sustain an autocrine loop whereby it promotes cytokine expression which in turn activates HA binding activity of receptors. HA has been reported to upregulate the expression of many of the factors that upregulate CD44 affinity for FIA (E I Maradny, E. et al Hum Reprod 12:1080 (1997); Turley, E. A. et al J. Biol Chem 277:4589 (2002)).

The effects of HA binding to PBMCs or B-cells are diverse and affect cytokine production; differentiation of blast and other cells; enhance recognition of tumor cells; promote CD3-mediated T cell activation; mediate adhesive, chemotactic and phagocytic responses; as well as regulate apoptosis and proliferation. It is therefore expected that enhanced expression of HA receptors on white cell populations will increase immune functions.

Several studies have reported that the elimination of radiolabeled HA, administered IV as a bolus dose, from the serum of sheep or rabbits follow first order or linear kinetics in elimination rates (Lebel, L. et al Pharm Res 6:677 (1989)). However, IV injection of HA to levels that are higher than in normal serum, results in non-linear elimination kinetics that are best modeled using Michaelis-Menten kinetics (Lebel, L. et al Eur J Clin Invest 24:621 (1994); Torsteinsdottir, I.T. et al Semin Arthritis Rheum 28:268 (1999); Lindqvist, U. et al Clin Chim Acta 210:119 (1992)). Prior studies assumed either a one compartment model of elimination (e.g. liver), or two compartment model of elimination (e.g. liver and to a minor extent kidney), and no significant interaction or pooling of HA in other tissues. Since sampling was acute (usually 1 hour) and frequent, these studies have provided the most reliable data to date for elimination of HA from human subjects.

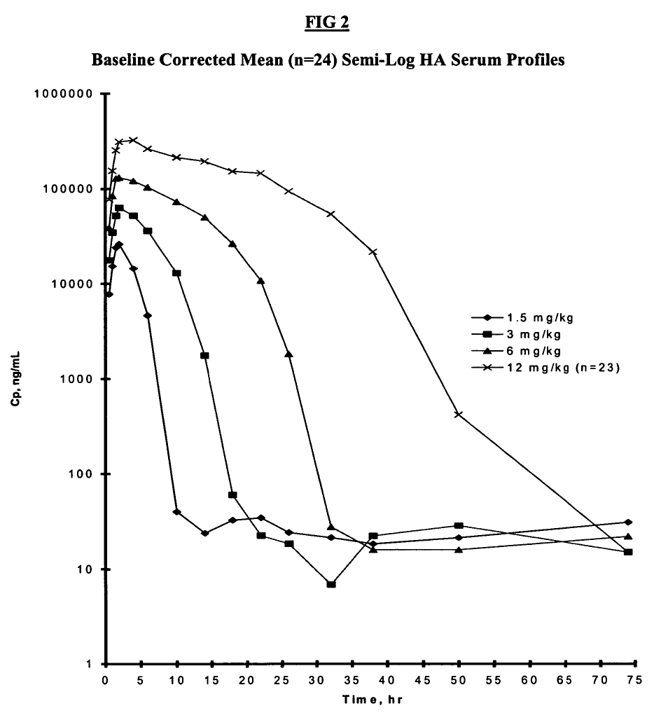

When the data are averaged and analyzed for all four dosing periods, assuming Michaelis-Menten kinetics and using a one-compartment model, a Vₘ similar to that reported in the previous studies was obtained. As shown in FIG. 2, data re-plotted as log-linear functions of serum HA levels vs. time demonstrates the complexity of each elimination curve with respect to slope changes. Analysis of the curves, disregarding changes in slope within a curve, clearly shows that elimination of HA from serum changes with increasing doses administered over time. Since slope changes are a qualitative indicator of changing elimination rates, this demonstrates that elimination kinetics of multiple doses of HA are much more complicated than either a one or two compartment model takes into account. Even when using a statistical moment model, obtaining Vₘ and Kₘ constants with Michaelis-Menten Kinetic analysis is still not appropriate, given the sparse sampling and the complexity of last two elimination curves. We therefore calculated Mean Retention Time (MRT); volume of solution, which is similar to volume of distribution; and clearance rates; for each curve using values after the infusion period and referred to in the following examples. These values allowed establishment that the rates of elimination were different for each dosing period, as determined by these parameters. In particular, the clearance rates and volume of solution decrease most abruptly in the last two dosing periods, whereas MRT decreases most abruptly in the last dosing period of 12 mg/kg.

While it is not intended that the present invention be limited to any particular mechanism by which the methods achieve a desired result, it is proposed that elimination of HA from serum is a complex process of pooling or retention followed by elimination. As such, the present invention is not intended to be limited to a specific range of molecular weights of HA. Though an understanding of the underlying mechanism is not necessary to practice the invention, one factor that might contribute to alternation of the elimination rate is the binding of exogenous HA to plasma proteins (Hayen, W. et al J Cell Sci 112:2241 (1999); Bost, F. et al Eur J Biochem 252:339(1998); Weigel, P. H. et al Ciba Found Symp 143:238 (1989)), to the vasculature (Savani, R. C. et al J Biol Chem 276:36770 (2001)) and within other tissues (Toole, B. P. et al J Biol Chem 277:4593 (2002)). In addition, increased binding to white blood cells, chondrocytes, endothelial cells, fibroblasts, breast epithelial cells, monocytes and macrophages can reasonably be considered to contribute to the alteration of the elimination rate. (Kabayashi, H. et al. Int J Cancer 102:379 (2002); Annabi, B. et al J Biol Chem 279:21888 (2004); Cichy, J. et al FEBS Lett 556:69 (2004); Lesley, J. et al J Biol Chem 275:26967 (2000); Maradny, E. et al Hum Reprod 12:1080 (1997); Turley, E. A. et al J Biol Chem 277:4589 (2002)). Plasma HA binding proteins include α-trypsin inhibitor and tissue HA receptors include HARE, CD44, RHAMM and LYVE-1/CSRSBP-1. HARE are endocytic receptors expressed on endothelial cells of the liver, while both CD44 and RHAMM are expressed on vasculature endothelium. Expression patterns of these HA receptors differ in large versus small vessels, which could add complexity to binding curves (Turley, E. A. et al J Biol Chem 277:4589 (2002); Lokeshwar, V. B. et al J Biol Chem 275:27641 (2000); Savani, R. C. et al J Biol Chem 276:36770 (2001)). LYVE-1/CSRSBP-1 is expressed in lymphatics (Charrad, R. S. et al Nat Med 5:669 (1999)), liver sinusoidal endothelium (Mouta Carreira, C. et al Cancer Research 61:8079 (2001)) and other tissues. Furthermore, plasma proteins such as α-trypsin inhibitor can be incorporated into HA-rich matrices within tissues. Therefore in addition to the complexity provided by an association of HA to multiple different binding proteins, potentially each with a different affinity and propensity for endocytic uptake, the reversible incorporation into tissues must also be taken into consideration.

Recent studies have demonstrated the non-linear adsorption or growth of HA incorporated onto borosilicate glass slides (Shi, M. et al J Immunol 167:123 (2001)) or damaged blood vessels (Jarvis, B. et al Am J Clin Dermatol 4:203 (2003)). In the first instance, the binding of HA to a protein is mimicked by including poly-lysine; while in the second by the presence of physiologically relevant receptors that have been documented to be expressed, particularly after injury, in blood vessels (Toole, B. P. Glycobiol 12:37R (2002); Savani, R. C. et al Int J Tissue React 17:141 (1995); Ward, M. R. et al Arterisocler Thromb Vasc Biol 21:208 (2001); Travis, J. A. et al Circ Res 88:77 (2001)). These studies conclude that a non-linear adsorption of HA to each surface is facilitated by the diffusion of the HA binding partner out of the substrate. This phenomena is clearly relevant to the blood vasculature as demonstrated by the second study and could contribute to both by the “diffusion” of serum HA binding proteins and by the release or shedding of HA receptors from tissue, including the endothelium. Taking this type of complex interaction into consideration permits kinetic analysis using more appropriate mathematical models.

Several studies have addressed whether reliable changes in one measure of elimination, the rate constant Vₘ, following a single or loading IV dose, can be used as a measure of active disease (Lindqvist, U. et al Clin Chim Acta 210:119 (1992); Hjerpe, A. et al Calcif Tissue Int 35:496 (1983)). To date, calculation of the Vₘ has not consistently identified patients with active arthritic or fibrotic disease, with the presence of high HA concentrations in serum the better indicator of active disease. The data disclosed herein indicates that the HA elimination from the serum during the penultimate and final dosing periods exhibits more complex regulation than the two previous periods. This complexity correlates with a transient increase in HA binding capability of white blood cells, as shown in Example 3. Since disease states such as malignancy, arthritis and fibrosis are accompanied and affected by altered HA receptor display, identifying and quantifying the complexity of elimination kinetics following multiple HA infusions will provide more useful clinical data to follow the activity of these diseases than other HA related methods.

HA preparations obtained from a commercial source did not contain detectable protein or DNA, as has been reported for some HA sources. Furthermore, molecular weight analysis of the final dosing period suggests that free HA fragments in serum are not generated, at least by the highest dose administered. HA fragments have been shown to be more bioactive in terms of promoting signaling pathways leading to cytokine or growth factor expression, which might lead to adverse effects to volunteers. In this study, no marked changes in synovial HA molecular weight occurred following administration of exogenous HA. These results together with the lack of toxicity seen in this study, indicate that repeated HA administered IV in doses of up to 12 mg/kg is safe for normal, healthy, humans.

The results disclosed herein indicate that there is a pooling of HA with increasing dose and/or repeated administration of HA, as reflected by the decreased volume of distribution and reduced clearance rate. These parameters are not linearly related to one another, indicating that multiple mechanisms are involved in the pooling of HA and its clearance from serum. Of particular note as shown in FIG. 2, the complexity of the slope of the curves increases with each dosing period. For example, the shoulder for each curve after the two hour infusion period is increasingly broad with increased dose and by 12 mg/kg shows several discernable changes in slope.

Though an exact understanding of the underlying mechanism is not necessary to practice the present invention, pooling or sequestration of HA could occur as a result of increased HA receptor expression. This could affect the diffusability of HA into tissues and also the affinity with which it binds to HA receptors. HA receptors, such as CD44, that bind to and can thereby sequester HA are expressed constitutively in most tissues; most notably in the liver and vascular tissue, particularly endothelial cells. Furthermore, several studies report altered HA receptor display in response to pharmacological doses of exogenously administered HA (Savani, R. C. et al Int J Tissue React 17:141 (1995)). The results indicate disclosed herein circulating cells heterogenous for HA binding activity early in the study. Exposure to HA over time activates an increasing number of these cells to acquire HA binding ability, and by the last dosing period, virtually all of circulating PBMC and B-cells have become activated in terms of their HA-binding ability. As such, HA binding by PBMC is a dynamic process that is subject to regulation by exogenous infusions of HA. Though the invention is not intended to be limited to one potential mechanism of action, these results indicate that sequestration of HA, observed by kinetic analysis, is mediated at least in part, by altered HA receptor binding. This modulation of receptors as a function of HA exposure, which includes but is not limited to time of HA presence and concentration, leads to the ability to modify receptor presence and/or availability in cell populations through selective prior administration of HA. As HA has been previously reported to upregulate the expression of many factors that effect the affinity of CD44/HA interactions (E I Maradny, E. et al Hum Reprod 12:1080 (1997); Turley, E. A. et al J Biol Chem 277:4589 (2002)); the present invention enables alteration HA binding to cells, and alteration of HA cellular receptor ability to bind HA, including white blood cells, chondrocytes, endothelial cells, fibroblasts, breast endothelial cells and hemotopoietic stem cells.

The results disclosed herein show that the elimination rates of HA from serum are strongly reduced with dose administration over time and our data is consistent with, though the invention is not limited to, a mechanism resulting in a pooling or sequestering of HA possibly as a result of increased HA binding to cells. Consistent with this hypothesis, we observe an increase in HA binding to white blood cells, demonstrated through observation of B-cells and monocytes, during the fourth and final dosing period. This leads to the ability to modulate and control the rate of clearance of HA in a human patient through prior administration of HA.

As taught by Canadian Patent 2,167,044 “Oral Administration of Effective Amounts of Forms of Hyaluronic Acid” (the '“044 Patent”); oral administration of HA results in an increase in blood levels equal to that observed by similar dosages administered subsequently. As taught by the '044 Patent oral administration of sodium hyaluronate, (M.W.—400,000 daltons, 2% by weight in sterile water), at a single dose of 30 mg/kg, results in an increase in serum hyaluronic acid on an order of that seen for the subcutaneous administration of this compound at 30-100 mg/kg. As oral HA administration results in an increase in HA blood levels; intravenous, oral and subcutaneous administration of HA will effect an increase in HA blood levels, as contemplated by the present invention.

Recent studies have demonstrated that changes in the quantity of HA in the extra cellular matrix of cells is responsible for detectable changes in magnetic resonance imaging, specifically that based upon apparent diffusion coefficient (ADC) imaging. (Sadejhi, N. et al Am J Roentgenol 181:235 (2003)). Therefore changes to the ability of cells, and the tissues to which they are member; to bind HA enables increased binding of HA to the cell/tissue. This results in a method to increase the ability of HA to bind to cell types, through the means and methods disclosed herein; for the purpose of imaging cells or tissues of interest through imaging methods including, but not limited to, magnetic resonance imaging and ADC magnetic resonance imaging. Particularly relevant uses of this method include, but are not limited to, imaging of B-cell lymphoma, breast tissue and other tissues sensitive to HA induced HA receptor affinity of HA receptor increase.

Further imaging improvements may be obtained through inclusion imaging enhancers or markers such as fluorine-19 associated with imaging HA administered to the patient through either covalent or ionic bonds, alone or as part of a molecule. Alternatively one may incorporate radioactive nuclides, associated with HA through either covalent or ionic bonds, alone or as part of a molecule.

HA can be labeled with a diagnostic agent using appropriate methods, such as linking a radioactive isotope to the HA molecule through covalent means; directly or through a molecular intermediate. For example, one may alter the molecules disclosed in U.S. Pat. No. 6,673,919 to Yui, to incorporate a diagnostic agent; followed by reaction with, and modification of, HA as taught therein.

Use of HA as a diagnostic is known in the art, for example as taught in U.S. Pat. No. 5,772,982 to Coward, which teaches the use of radioactive isotopes as diagnostic agents in association with hyaluronic acid in association with positron emission tomography (PET); scintigraphy, such as gamma camera scintigraphy; nuclear magnetic resonance imaging (MRI); tomography, including computerized tomography (CT), PET, and single photon emission computed tomography (SPECT); and various forms of radiography including mammography, xeroradiography, cerebral arteriography, angiography, digital subtraction angiography and iodine K-edge dichromography. Coward further teaches the use of specific radioactive isotopes, with sufficient energy levels to allow detection, with preferred isotopes including americium-241, barium-137 (particularly in barium sulfate), calcium-47 (particularly in calcium chloride), cesium-137 (particularly in cesium sulfate and cesium chloride), chromium-51 (particularly in sodium chromate, chromium disodium edetate, or chromic chloride), cobalt-60 and cobalt-57 (in their metallic form or in vitamin B.sub.12), copper-64 (particularly in copper versenate), fluorine-18 (particularly in sodium fluoride), gallium-67 (particularly in gallium citrate), colloidal gold-198, colloidal indium-113m, indium-ill (particularly in indium chloride), iodine-123 and iodine-125 (particularly in sodium iodide and iodohippurate sodium), iodine-131 (particularly in sodium iodide, diiodofluorescein, iodohippurate sodium, sodium diatrizoate, iodopyracet, diatrizoate methyl glucamine, sodium diprotrizoate, sodium acetrizoate, or sodium iothalamate), iridium-192, iron-55 and iron-59 (particularly in ferrous citrate, ferrous sulfate and ferric chloride), krypton-85 gas, lead-210, mercury-197 and mercury-203 (particularly in chlormerodrin), phosphorus-32 (particularly in sodium phosphate), potassium-42 (particularly in potassium carbonate), radium-226, ruthenium-106, selenium-75 (particularly in seleno-methionine), sodium-24 (particularly in sodium chloride), strontium-85 and strontium-87m (particularly in strontium nitrate or strontium chloride), sulfur-35 (particularly in sodium sulfate), technetium-99m (particularly in pertechnetate, technetium DTPA, technetium stannous polyphosphate, technetium stannous etidronate or colloidal technetium sulfate), thallium-201 (particularly in thallous chloride), tritium, xenon-133 gas, and ytterbium-169 (particularly in ytterbium-DTPA). Coward teaches that naturally occurring elements that are constituents of organic matter, including carbon, oxygen and nitrogen, can be made radioactive and thus capable of detection using PET. Finally, Coward teaches that stable isotopes such as carbon-13, phosphorous-13, boron-11 and fluorine-19 can be used for MRI.

Use of the present invention will result in increased sensitivity for, and utility of, HA as a diagnostic through the presently enabled ability to generally or specifically increase a given cell population's and/or tissue's affinity or binding capacity for HA. Elements emitting a signal detectable by an imaging apparatus, such as radionuclides and fluorescent molecules; isotopes capable of differentiation through magnetic resonance imaging; or unmodified HA detected by magnetic resonance, may be utilized as a “marker” to identify the presence of HA in a patient. HA containing such a marker, administered following systemic or localized administration of sequential dosages of HA, will have increased interaction with cells or tissues in which the presence or affinity of HA receptors is increased; thereby improving and better enabling HA based diagnostic imaging.

### EXAMPLES

The following examples are intended to illustrate, though not limit, the scope of the present invention.

Materials and Methods

The following are the procedures and materials used for the accompanying examples except where otherwise described.

Antibodies and Reagents

FMC63 (CD19) antibody was conjugated to phycoerythrin (PE). LeuM3-PE (CD14), Leu3-PE (CD4) and Leu2-PE (CD8) were obtained from Becton Dickinson (San Jose, Calif.). Isotype matched control monoclonal antibodies (mAb) were obtained from Southern Biotech (Birmingham, Ala.). For infusions, lyophilized HA (medical grade, Hyal Pharmaceutical Corp., Mississauga Ontario) was dissolved in Phosphate Buffered Saline (PBS) and autoclaved for sterility and for reduction of molecular weight. Although the HA samples used for this study were not analyzed for their initial molecular weight, samples from the same company were analyzed for molecular weight using HPLC/SEC multi-angle laser light scattering (Lifecore Biomedical, Minnesota). For cell binding assays, Healon® HA of medical grade was obtained from Pharmacia (Dorval Quebec) and conjugated to fluorescein (FITC) as described previously (Masellis-Smith, A. et al Blood 87:1891 (1996)).

Assessment of HA Purity

HA preparations were tested for the presence of protein, DNA and endotoxin. Protein content was assayed by absorption at 280 nm and the presence of DNA was determined by electrophoresis of 10-100 μg HA in agarose gels containing 0.7% agarose and 0.5μg/ml ethidium bromide at 100 V for 3 hours. DNA was visualized using a UV transilluminator (wavelength: 302 nm) (Filion, M. C. et al J Pharm Pharmcacol 53:555 (2001)). Endotoxin was detected using a colorimetric Limulus ameobocyte lsyate assay with a sensitivity of 0.01 endotoxin units/ml (Lindqvist, U. et al Clin Pharmacokinet 41:603 (2002)).

Injection of HA and Sampling

Between 8-10 AM of day one of the study, blood was drawn from volunteers 1-2 hrs prior to HA infusion. Twenty-four volunteers were then infused to 1.5 mg/kg with medical grade 1% HA (Hyal Pharmaceutical Corp. Mississauga, Ontario) over 2 hours. Blood was collected in 1×1.8 ml citrated vacu-containers. Samples were drawn at 0.5, 1 and 2 hours during the 2 hour infusion (dosing period) and at 2, 4, 6, 10, 14, 18, 22, 32, 38 50 and 74 hours post-infusion. Samples drawn during this period were classified as period 1. At seven-day intervals, volunteers were infused with 3.0 mg/kg (period 2), 6.0 mg/kg (period 3) and 12 mg/kg (period 4) following the above schedule of sampling. One volunteer displayed flu-like symptoms during the second infusion period and was withdrawn from the study.

Assays

Sub-groups of volunteers were sampled for platelet aggregation assays, HA molecular weight analyses and HA binding to T-cells, B-cells and monocytes. For platelet aggregation and molecular weight assays, the regular blood draws for the 0.5 and 1.0 hour of the dosing period and the 42 hour post-infusion time-points were eliminated. 2×1.8 ml blood draws were taken during period 4 for sub-group A (volunteers #7-10) at 1 hour pre-dose, 0.5 hour during the dosing period and 48 hour post-infusion; while draws for sub-group B (volunteers #11, 12, 13, 18) were taken during period 4 at 2 hours pre-dose, 1.5 hours during the dosing period and 72 hours post-infusion. Clotting times and aggregation of platelets did not differ throughout this sampling period. The serum samples obtained from sub-group B was analyzed for HA molecular weights as described below. For analysis of FITC-HA binding, blood was drawn from six selected volunteers from each period and draws were analyzed for cell size and granularity, followed by cell surface phenotyping to identify CD4⁺, CD8⁺ T-cells, B-cells and monocytes, as described below. Data for 6 individual donors, from PBMC taken at 6 time points (0, 1, 4, 12, 24, 48 and 72 hours) were collated and the area under the curve (AUC) calculated for each dose of HA.

Measurement of Serum HA

The analysis for HA in serum samples from 23 healthy volunteers for each time point was performed using a Pharmacia HA Test (Pharmacia, Inc. Uppsala, Sweden). The limit of quantification (LOQ) for the assay was 10 ng/mL, and the limit of detection (LOD) was 4.73 ng/mL.

Pharmacokinetic Analysis:

Serum HA levels for each subject and each treatment were initially analysed assuming a one compartment, Michaelis-Menten model and averaging all four doses for kinetic analysis. Pharmacokinetic parameters were calculated for HA using PKCALC, a non-compartmental pharmacokinetic data analysis software (PKCALC, Wallace Laboratories, Princeton, N.J., 1993) (Shumaker, R. C. Drug Metab Rev 17:331 (1986)). Since all of the subjects had a baseline value of HA due to endogenous levels of the compound, the serum concentrations were corrected for baseline prior to pharmacokinetic analysis of the data. The following pharmacokinetic parameters were calculated from the serum data after baseline correction: “Cₘₐₓ” maximum serum concentration (ng/mL); “Tₘₐₓ” time to maximum plasma concentration (h); “AUC₀₋ₜ” area under the concentration-time curve (ng·h/mL) from 0 to the last time point (usually 72 hrs) with a quantifiable concentration of HA (this value was calculated using the linear trapezoidal rule); “AUC₂₋ₜ” area under the concentration-time curve (ng·h/mL) from the end of the infusion (2 hrs) to the last time point (72 hrs) with a quantifiable concentration of HA (this value was calculated using the linear trapezoidal rule); “Kₘ”, the Michaelis-Menten constant or the dissociation constant for the enzyme-drug complex (μg/mL); “Vₘ” the maximum velocity of the metabolic reaction, Vₘ divided by volume of distribution of HA (μg/mL·h). Results are presented as the mean of 23 subjects after all four doses. Since all of the subjects had a baseline HA value due to endogenous levels of the compound, the serum level results were corrected for baseline prior to pharmacokinetic analysis of the data. For each subject, the concentration at zero hour (endogenous HA) was subtracted from each other concentration of the same subject and the concentration differences termed “C-C₀.” Pharmacokinetic calculations were conducted using the individual baseline corrected data and non-compartmental analysis. The areas under the C-C₀ versus time curves were calculated using the trapezoidal rule. Data were also analyzed using a Statistical Moment model of Michaelis-Menten kinetics and for these analyses rate constants (see Table 5) were derived for each dose.

Molecular Weight Analysis:

HA and other glycosaminoglycans were precipitated with cetyl pyridinium chloride from the serum samples of the volunteer sub-groups A and B (Hjerpe, A. et al Calcif Tissue Int 35:496 (1983)). The precipitates were dissolved in Phosphate Buffered saline (PBS) then chromatographed using Sephadex 200 columns that were standardized using dextran standards (average molecular weight: 11,300-2,000,000 Da). 1 ml fractions of a total volume of 120 ml were analyzed for uronic acid using a modified anthrone assay (Somani, B. L. et al Anal Biochem 167:327 (1987)). The molecular weights were determined using non-dissociative elution conditions and were grouped for convenience into sets of: greater than 2×10⁶ Da, between 1.85×10⁵ to 2×10⁶ Da, and less than 1.85×10⁵ Da. Values represent the mean of N=6±S.E.M.

Measurement of HA Binding by Subsets of Peripheral Blood Mononuclear Cells (PBMC)

PBMC were purified by centrifugation over FICOLL PAQUE® (Pharmacia, Dorval, Quebec), washed and stained for surface marker expression as well as for the ability to bind FITC-HA as previously described (Masellis-Smith, A. et al Blood 87:1891(1996); Pilarski, L. M. et al Blood 93:2918 (1999); Sadeghi, N. et al Am J Roentgenol 181:235 (2003)). To determine the ability of PBMC subsets to bind HA, PBMC were stained in two color immunofluorescence with PE-coupled mAb to a defined surface marker and HA-FITC. The optimum amount of mAb-PE and of HA-FITC was determined by titration using PBMC from untreated normal donors. HA binding and T-cells (CD4⁺ or CD8⁺), B-cells (CD19⁺) or monocytes (CD14⁺) was analyzed in replicate aliquots of each PBMC sample. Blood samples were taken over a time period of 72 hours post i.v. infusion of each dose of HA. Each mAb was compared to an isotype matched control antibody to exclude any staining due to background. Data for 6 individual donors was obtained for PBMC taken at 6 time points (0, 1, 4, 12, 48, and 72 hours).

PBMC stained with mAb-PE and FITC-HA were analyzed by flow cytometry using a FACS sort (Becton Dickinson, Oakville, Ontairo). Files of 10,000 cells were collected after exclusion of red and dead cells, and were analyzed using LysisII software; (Becton Dickinson, Oakville, Ontairo). Electronic gates were set to include those cells expressing CD4, CD8, CD19 or CD14, according to the mAb used to stain the aliquot of PBMC, and the staining with FITC-HA on that subset plotted as a histogram. Auto-fluorescence of PBMC, and PBMC stained with IgG-FITC or avidin-FITC, were used as negative controls to evaluate staining by FITC-HA. Staining was moderately bright for FITC-HA for 13 untreated normal donors, with mean fluorescence intensity in arbitrary units of 13.3 for CD4⁺ T-cells of 11.2 for CD8⁺ T-cells, of 26.4 for CD19⁺ B-cells and of 70.2 for CD14⁺ monocytes. The HA binding by subsets of PBMC after infusion of HA was compared to the normal values.

### Example 1

Characterization of HA

Several HA preparations, from same source batches used in this study were analyzed for molecular weight polydispersity. Values for two HA samples re-constituted in PBS and heat sterilized are given in Table 1, along with the molecular weight of a sample not subjected to heat sterilization. All samples were polydisperse in molecular weight as is characteristic of HA, with an average molecular weight ranging from 276-289 kDa with heat sterilization, and 509-750 kDa without. Samples were also analyzed for protein contamination. As shown in Table 2, there was no significant protein present in these samples. One report has noted DNA contamination in several commercial samples of HA and further shown that DNA, and not HA, was responsible for the ability of samples to promote cytokine expression in monocytes in vitro (Lindqvist, U. et al Clin Pharmacokinet 41:603 (2002)). Therefore, the presence of DNA was also assayed for and as shown in Table 2, none was detected. Endotoxin was also not detected.

TABLE 1
Molecular weights and polydispersity of HA batches

| | | | | |
|---|---|---|---|---|
| | Average | | | |
| | Molecular | 80% Molecular Weight | | Poly- |
| Sample | Weight (kDa) | Distribution (kDa) | Radius | dispersity |

LYOPHIZED

| | | | | |
|---|---|---|---|---|
| a. | 519.7 | 167-1,290 | 74.1 | 1.289 |
| b. | 750.4 | 240-1,970 | 92.4 | 1.321 |

HEAT STERILIZED

| | | | | |
|---|---|---|---|---|
| c. | 276.7 | 106-578 | 53.9 | 1.221 |
| d. | 288.8 | 124-567 | 55.9 | 1.186 |

TABLE 2
Characterization of autoclaved HA

| Sample | Uronic acid/glucosamine | Protein | DNA | Endotoxin |
|---|---|---|---|---|
| c. | 1.0 | Not detec. | Not detec. | Not detec. |
| d. | 1.0 | Not detec. | Not detec. | Not detec. |

Although one subject dropped out due to flu-like symptoms, no adverse effects that could be linked to HA administration were observed doing the course of the study.

### Example 2

Kinetic Analysis of HA Elimination From Serum

The design of this study was based upon standard methods for obtaining an optimal dosing regime commonly used for bioactive drugs (Thompson, G. A. et al Drug Metab Rev 21:463 (1989)). An escalating dosing regime of HA, same source of which were analyzed in Example 1, was given over a period of 4 weeks with a weekly interval, judged from previous studies (Torsteinsdottir, I. T. et al Semin Arthritis Rheum 28:268 (1999)) to be long enough to allow elimination of serum HA prior to the next dose. Since the concentrations of HA administered IV in the present study were higher than those of previous reports using a bolus dose (Lindqvist, U. et al Clin Chim Acta 210:119 (1992)), this was judged to be a safer method of administration. The sampling period was also different as compared to previous studies. In the present study, samples were taken prior to HA administration, mid-infusion and at 11 time points between 2-72 hours (post-infusion). In contrast, other studies report more intense sampling over 1 or 2 hr periods following a single bolus intravenous (IV) injection of HA (Lebel, L. et al Eur J Clin Invest 24:621 (1994); Espallargues, M. et al Int J Technol Assess Health Care 19:41(2003)). In the present study, HA in serum samples taken at the times shown in Table 3, were averaged and corrected for baseline (non-treated) levels of endogenous serum HA. The means and standard deviations of the means for these samples are listed in Table 3.

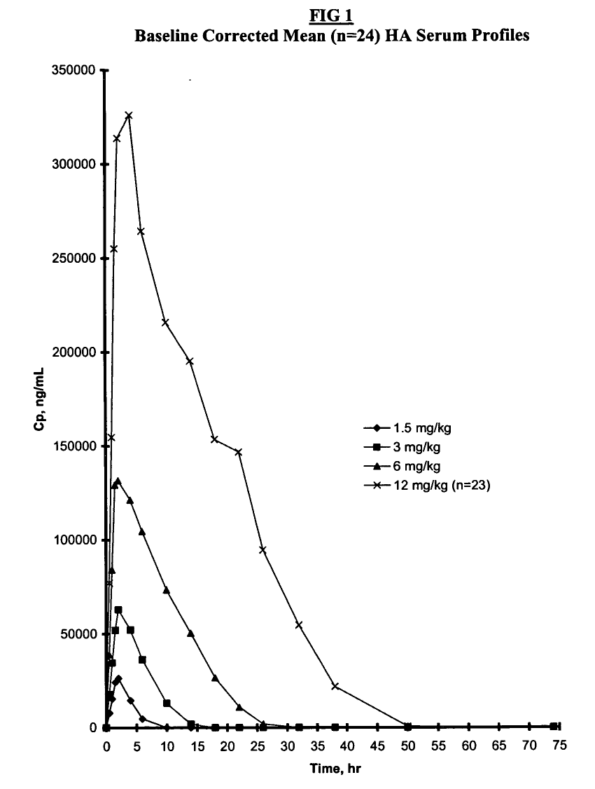

As shown in FIG. 1, for initial analysis, the baseline-corrected mean serum concentrations versus time for all four doses were plotted against HA concentration; demonstrating that the elimination of all four doses of HA is not linear over time. Since previous studies have modeled elimination rates assuming Michaelis-Menten kinetics, data was initially analyzed using a one-compartment model and averaging of the values for all of the doses, including the infusion period. The Kₘ given as a K_{m′}, does not include the volume of distribution (as this was not measured) therefore is not directly comparable to the Km of previous studies. Table 4 shows the kinetic parameters when values for all 4 doses were averaged. A Vₘ of 11.05 μg/mL/h (or 0.184 μg/ml/min) and K_{m′} of 40.86 μg/mL were obtained from the averaged data. The Vₘ is within the range of values reported for healthy volunteers given a single bolus IV injection (Espallorgues, M. et al Int J Technol Assess Health Care 19:41(2003)).

TABLE 3
Baseline Corrected HA, Mean Cₚ, ng/mL

| | 1.5 mg/kg | 3.0 mg/kg | 6.0 mg/kg | 12.0 mg/kg |
|---|---|---|---|---|
| Time, h | Mean (±SD) | Mean (±SD) | Mean (±SD) | Mean (±SD) |
| 0 | 0 | 0 | 0 | 0 |
| | (0) | (0) | (0) | (0) |
| 0.5 | 7761 | 17713 | 38838 | 77186 |
| | (1130) | (3021) | (6162) | (11297) |
| 1 | 15280 | 34625 | 84083 | 154713 |
| | (2783) | (5621) | (13648) | (26503) |
| 1.5 | 24052 | 52052 | 129204 | 254994 |
| | (3653) | (9039) | (16063) | (40017) |
| 2 | 26284 | 63063 | 131549 | 313554 |
| | (3918) | (16099) | (17324) | (41271) |
| 4 | 14419 | 52167 | 121122 | 325913 |
| | (3384) | (7646) | (13000) | (51980) |
| 6 | 4633 | 36334 | 104426 | 264232 |
| | (3518) | (6390) | (15384) | (40440) |
| 10 | 40 | 12934 | 73627 | 215500 |
| | (56) | (7139) | (12514) | (28998) |
| 14 | 24 | 1755 | 50440 | 195145 |
| | (12) | (3188) | (13547) | (32153) |
| 18 | 33 | 61 | 26522 | 153467 |
| | (19) | (103) | (13128) | (22857) |
| 22 | 35 | 23 | 10769 | 146773 |
| | (7) | (14) | (12496) | (27505) |
| 26 | 24 | 19 | 1839 | 94579 |
| | (16) | (10) | (4367) | (31429) |
| 32 | 22 | 7 | 28 | 54615 |
| | (17) | (4) | (55) | (29332) |
| 38 | 18 | 23 | 16 | 21795 |
| | (10) | (14) | (11) | (21939) |
| 50 | 22 | 29 | 16 | 419 |
| | (14) | (10) | (11) | (1373) |
| 74 | 31 | 15 | 22 | 15 |
| | (12) | (10) | (16) | (7) |

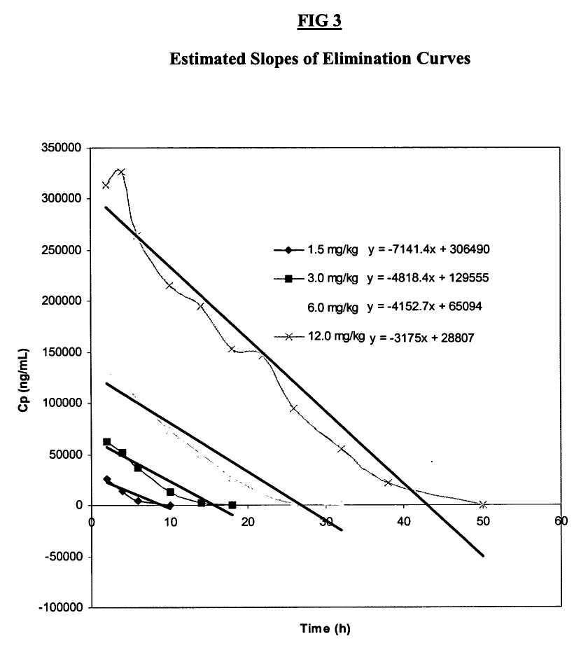

To assess whether or not elimination kinetics of serum HA change with escalating dose, and thus evaluate whether averaging all of the data to obtain kinetic constants is appropriate for the study design, any dose differences were amplified by replotting data as log-linear functions of serum HA concentrations versus time as shown in FIG. 2. Average slopes for each elimination curve were assessed by forcing curves through a linear line. As shown in FIG. 3, the estimated values for the slope change with each dosing period, particularly for the last dose (12 mg/kg). These results indicate that averaging values from each dosing period will obscure important differences between each dose and therefore further kinetic constants were calculated for individual doses separately. FIG. 2 shows that HA serum levels for each dosing period decreased to approximate baseline levels (zero) but never to zero, although this range was close to, or at, the assay limits of accurate detection for HA.

To improve accuracy of calculations, the infusion period was eliminated from analyses. The analysis period for each dose was chosen to be when serum HA concentration was the highest, to when values were first observed to be at or near the limit of the accuracy of the assay (baseline HA levels). This period was between 2-14 hrs for 1.5 mg/kg dose; 2-22 hrs for 3 mg/kg dose; 2-32 hrs for 6 mg/kg dose and 2-74 hrs for 12 mg/kg dose. For this analysis, Mean Retention Time (MRT, hour), Volume of Solution (designated as Volume of Distribution (VD) since it approximates volume of distribution in blood, assuming volume=4.4% of body mass) and Clearance Rate (CR, ml/hr) were calculated; to provide quantification of the elimination kinetics for each dose. Values are given in Table 5.

TABLE 4
Mean Pharmacokinetic Parameters for HA Obtained After Four Intravenous Doses (n = 23)

| | 1.5 mg/kg | 3.0 mg/kg | 6.0 mg/kg | 12.0 mg/kg* |
|---|---|---|---|---|
| Parameter | mean (SD) | mean (SD) | mean (SD) | mean (SD) |
| Cₘₐₓ (ng/mL) | 26688 (3661) | 65965 (12775) | 139523 (14807) | 333146 (48052) |
| Tₘₐₓ (h) | 1.90 (0.21) | 2.06 (0.63) | 1.75 (0.26) | 3.22 (1.00) |
| AUC₀₋ₜ (ng · h/mL) | 99610 (22398) | 401877 (83906) | 1499406 (262488) | 5923375 (1038514) |
| AUC₂₋ₜ (ng · h/mL) | 69492 (19342) | 333916 (76300) | 1340457 (257495) | 5599561 (1005755) |

*n = 22
Calculated Michaelis-Menten Constants:
Vₘ, = 11.05 μg/mL · h
Kₘ = 40.86 μg/mL

TABLE 5
Kinetic Parameters for Clearance Rates During Each
Dosing Period Using a Statistical Moment Model

| Dose (mg/kg) | MRT (hr) | VD (mL) | CR(mL/hr) |
|---|---|---|---|
| 1.5 | 3.66 | 79.37 | 21.67 |
| 3.0 | 5.42 | 48.49 | 17.89 |
| 6.0 | 8.39 | 30.67 | 3.65 |
| 12.0 | 14.65 | 31.36 | 2.14 |

The MRT for the second (3 mg/kg) and third (6 mg/kg) dosings increase by 50% with each dose doubling while the MRT for the final dose (12 mg/kg) is increased by 74%. The VD decreases by approximately 60% for each dose from 1.5 to 6.0 mg/kg but plateaus at 6.0 mg/kg dose so that the final two doses are similar. A decrease in CR is also observed although the difference between the first and second dose is small (20%), but a four-fold drop in the CR occurs between the second and third dose. There is a further 70% decrease in the CR for the final dose (12 mg/kg) of HA.

Since blood samples were being routinely taken throughout this study and since blood cells including T-cells, B-cells and monocytes have been reported to bind to HA and to express HA receptors such as CD44 and RHAMM, cells were analyzed for their ability to bind to FITC-HA using FACS analysis to detect binding and assessing binding for each dosing period.

### Example 3

HA Binding to Blood Cells

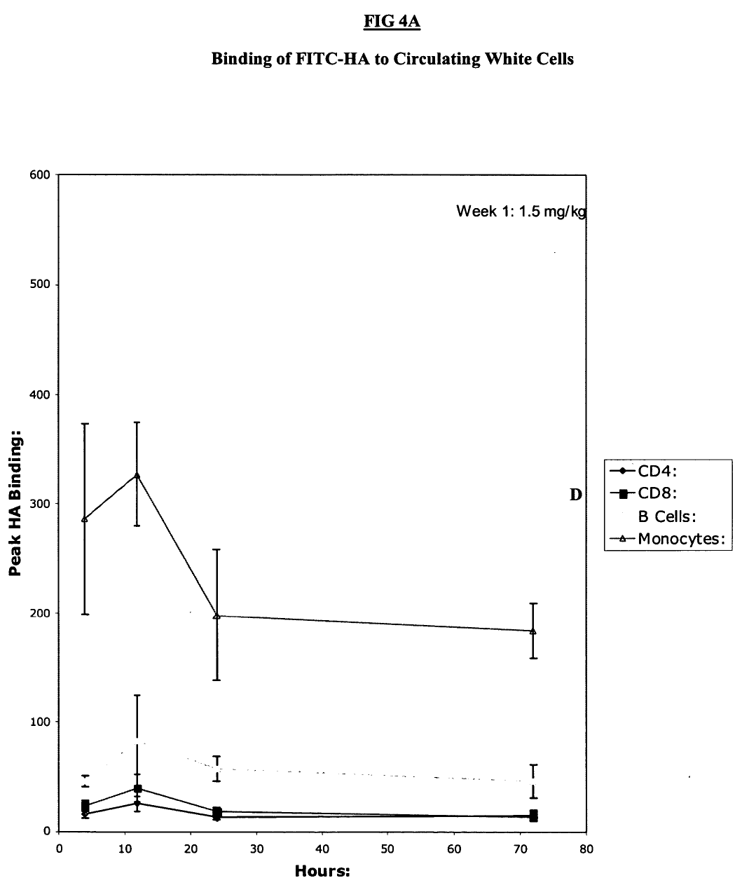

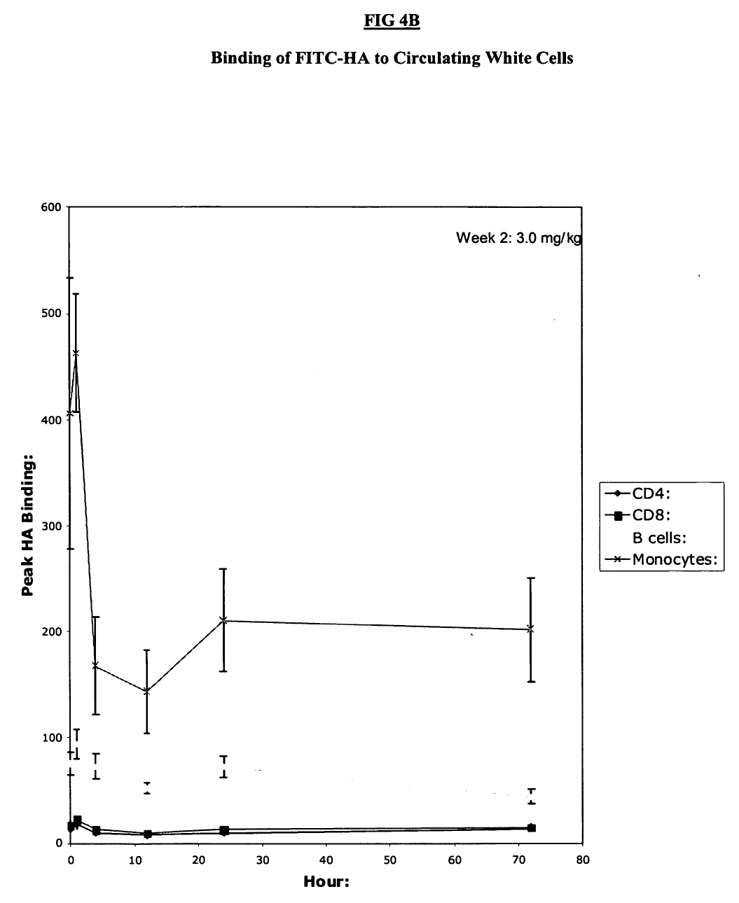

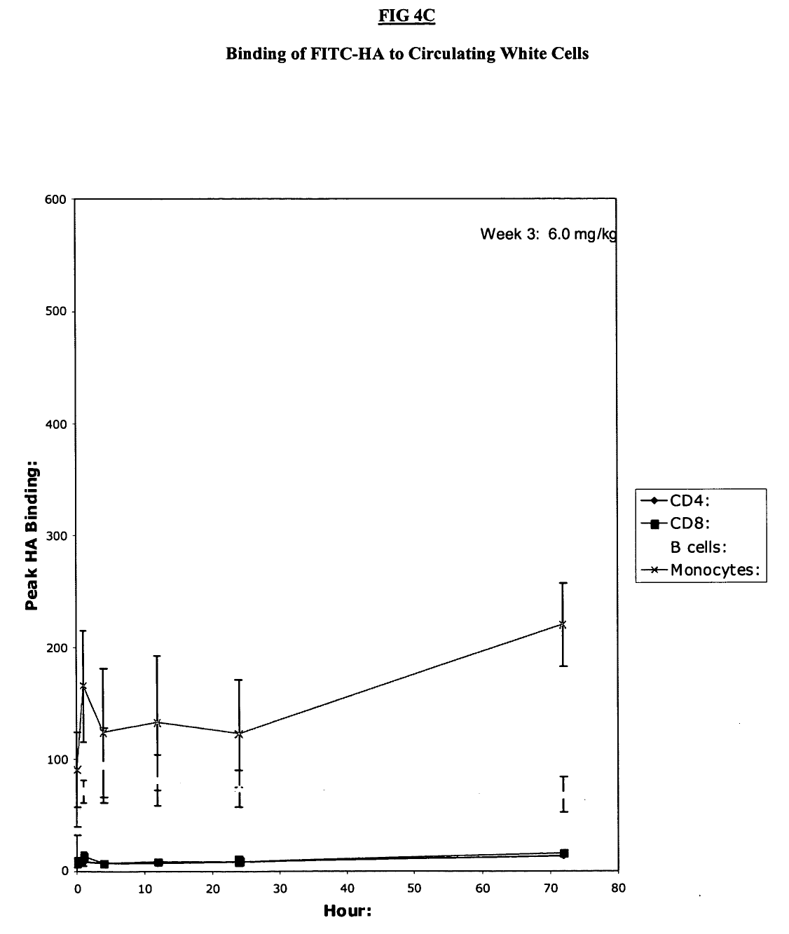

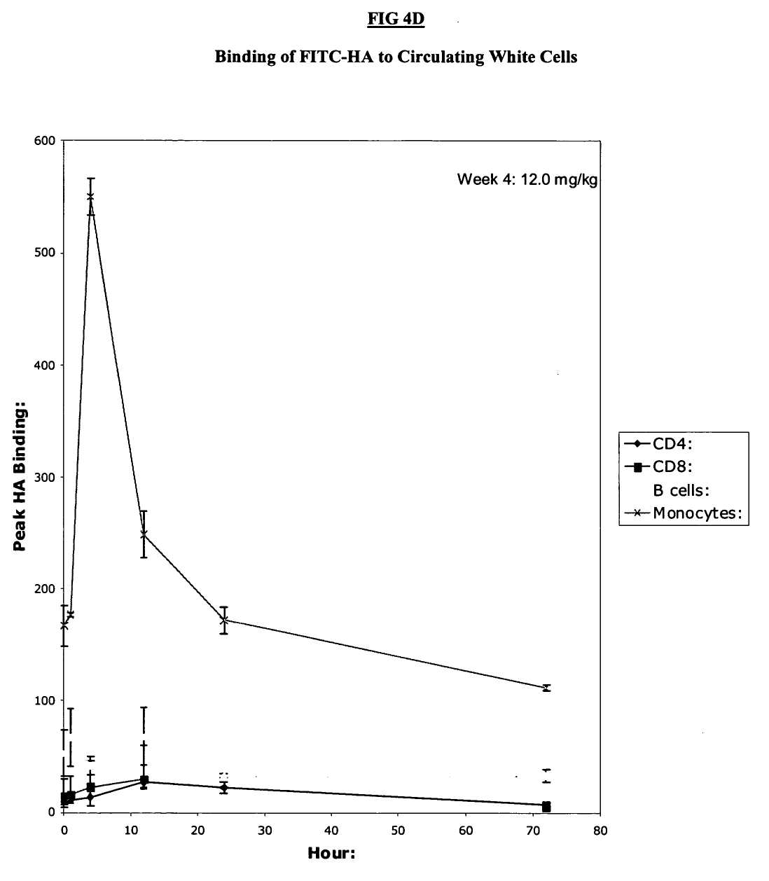

The HA-binding capacity of T-cells (CD4⁺ and CD8⁺), B-cells (CD 19⁺) and PBMC (CD14⁺) were analyzed by multiparameter flow cytometry. The flow cytometric assay measures HA binding by individual PBMC, with intensity of green fluorescent staining indicating the extent of HA binding by each cell. HA binding by a given subset of cells is quantified as the number of orange mAb-stained PBMC that are also stained green through binding of HA-FITC to the cell surface. All cell types bound FITC-HA, with PBMC binding to a greater extent than B-cells, and T-cells (both CD4⁺ and CD8⁺) bound the least as shown in FIG. 4. Modulation of HA binding occurred for all cell types, but this was particularly obvious during the fourth dosing period as shown in FIG. 4A-D, and was most dramatic for PBMC and B-cells.

In the first dosing period there was a trend, but not statistically significant, (Student's “t” test) for all cell types to exhibit a transient increase in HA binding at 12 hrs FIG. 4A. During the second dosing period, all B-cell and PBMC subsets exhibited a significant increase in HA binding at 1 hour post-infusion of HA, followed by a significant decrease to baseline values at 4 hours. Levels of binding for B-cells and monocytes increased again by 24 hrs post-infusion and for monocytes stabilized at the levels observed during the first infusion period. During the third dosing period, a trend, though not statistically significant (Student's “t” test) towards increasing HA binding occurred between 24 and 72 hours. However, the fourth dosing period resulted in a three-fold and statistically significant, transient increase (significant at p<0.0001, Student's “t” test) in binding of FITC-HA in monocytes and B-cells between 4-12 hrs and 12-20 hrs respectively.

Importantly, the variability in binding, as deduced by standard deviations of the mean, large in the first two dosing periods, was reduced in the third dosing period and dramatically decreased in the fourth dosing period.

The binding of HA to its receptors has been reported to depend, in part, upon the molecular weight of HA and also the biological consequences of HA binding can be molecular weight dependent (Day, A. J. et al J Biol Chem 227:4585 (2002)). For example, HA fragments of less than 30,000 Da have been reported to promote apoptosis of tumor cells and to promote angiogenesis (Slevin, M. S. et al J Biol Chem 277:41046 (2002)). Therefore, the molecular weight changes in serum HA following IV administration during one dosing period were assessed, and compared that to the molecular weight of endogenous HA present in serum prior to IV administration of HA.

### Example 4

Molecular Weights of Serum HA

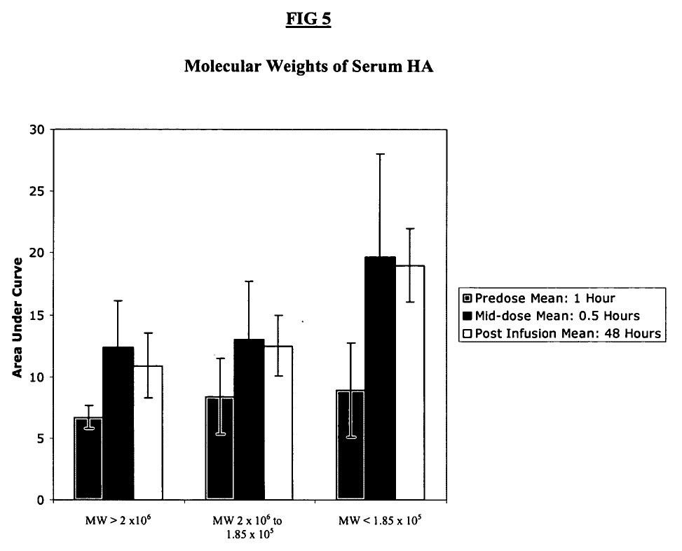

The molecular weight of HA in the serum of four volunteers were sampled at 2 hours prior to IV infusion, 1.5 hrs during the dosing period and 72 hours post-infusion of HA (12 mg/kg). As shown in FIG. 5, a significant amount of HA from all samples chromatographed at greater than 2×10⁶ Da. This is higher than the original HA at administration as shown in Table 1. This discrepancy is likely due to the different methods used for molecular weight determination for the original HA preparation, as compared to that used for serum HA, and the use of non-dissociative conditions used during column chromatography (Schmidt, A. et al Physiol Chem 365:445 (1984); Salisbury, B. G. et al J Biol Chem 256:8050 (1981)). Furthermore, it is likely that the large molecular weight HA represents HA associated with protein complexes (Rogers, H. J. Fed Proc 25:1035 (1966); Oegema, T. R. et al J Biol Chem 256:1015 (1981)). The high molecular weight fraction increased at mid-infusion of exogenous HA, as expected, and decreased by 72 hours post-infusion. Although there was a trend towards an increase in smaller HA polymers and fragments as shown in FIG. 5, the differences were not statistically different (Student's “t” test).

### Example 5

Improved Use of HA as an Imaging Diagnostic

As a means to image cells or tissues, HA is administered to a patient, sequentially, so as to increase the ability of the tissue of interest to bind HA. One such method of sequential administration is disclosed in Example 2. Such administration may comprise sequential administration of dosages of HA, systemically or locally administered, such that there is a transient increase in HA presence in or adjacent to the cell or tissue. Imaging of the cells or tissue of interest is conducted, such that the increased binding of HA by the cells or tissue assists imaging. One non-limiting example of such imaging is use of ADC magnetic resonance imaging, in which altered ADC signal arises from the increased binding of, and therefore increased presence of, extra cellular HA about the cell or tissue of interest.

### Example 6

Improved Use of Modified HA as an Imaging Diagnostic

As a means to image cells or tissues using HA modified to contain, or otherwise containing a diagnostic agent; unmodified HA is administered to a patient, so as to increase the ability of the cells or tissue of interest to bind HA. Such administration comprises sequential administration of dosages of HA, systemically or locally, such that there is a transient increase in HA presence in or adjacent to the cell or tissue. An amount of HA, modified to contain, or otherwise containing a diagnostic agent, is administered to the patient and allowed to interact with the cell or tissue of interest. Using as a diagnostic agent, 99^{m}Tc-labeled stannous polyphosphate complexed with HA, imaging of the cell or tissue of interest is performed using a scintillation camera with appropriate collimator.

Although the above disclosure describes and illustrates various embodiments of the present invention, it is to be understood that the invention is not to be limited to these particular embodiments. Many variations and modifications will now occur to those skilled in the art. For a full definition of the scope of the invention, reference is to be made to the appended claims.

What is claimed is:

1. A method to alter elimination kinetics of hyaluronic acid (HA) in a patient comprising:
initial administration of HA to the patient sufficient to result in a transient increase in blood HA presence; and
at least one subsequent administration of HA to the patient sufficient to result in a transient increase in blood HA presence.

2. The method of claim 1, wherein there are 3 subsequent administrations.

3. The method of claim 2, wherein HA is administered in sequential dosages of 1.5 mg/kg, 3 mg/kg, 6 mg/kg and 12 mg/kg.

4. The method of claim 3, wherein there is a period of seven days between each of the subsequent HA administrations.

5. The method of claims 1 through 4, wherein HA is administered by means selected from the group consisting of intravenous injection, oral administration and subcutaneous injection.

6. A method to increase the ability of cells to bind HA comprising a multiplicity of HA administrations to the cells or cell environment

7. The method of claim 6 wherein the cells are in vivo.

8. The method of claim 7 wherein the multiplicity of HA administrations comprises
initial administration of HA to a patient sufficient to result in a transient increase in blood HA presence
at least one subsequent administration of HA to the patient sufficient to result in a transient increase in blood HA presence.

9. The method of claim 8, wherein there are three subsequent administrations.

10. The method of claim 9, wherein HA is administered in sequential dosages of 1.5 mg/kg, 3 mg/kg, 6 mg/kg and 12 mg/kg.

11. The method of claim 10, wherein there is a period of seven days between each of the subsequent HA administrations.

12. The method of claims 8 through 11, wherein HA is administered by means selected from the group of means consisting of intravenous injection, oral administration and subcutaneous injection means.

13. The method of claims 7 through 12 wherein said cells are selected from the group consisting of monocytes, macrophages, B-cells, CD4⁺ cells, CD8⁺ cells, white blood cells, chondrocytes, endothlelial cells, fibroblasts, breast endothelial and hematopoietic stem cells.

14. A method to increase the presence or affinity of cellular HA receptors comprising a multiplicity of HA administrations to the cells or cell environment.

15. The method of claim 14 wherein the cellular HA receptors are in vivo.

16. The method of claim 15 wherein the multiplicity of HA administrations comprises administration of HA to a patient sufficient to result in a transient increase in blood HA presence; and
at least one subsequent administration of HA to the patient sufficient to result in a transient increase in blood HA presence.

17. The method of claim 16, wherein there are 3 subsequent administrations.

18. The method of claim 17, wherein HA is administered in sequential dosages of 1.5 mg/kg, 3 mg/kg, 6 mg/kg and 12 mg/kg.

19. The method of claim 18, wherein there is a period of seven days between each of the subsequent HA administrations.

20. The method of claims 16 through 19, wherein HA is administered by means selected from the group of means consisting of intravenous injection, oral administration and subcutaneous injection means.

21. The method of claims 16 through 20 wherein said cellular HA receptors are located on the cells selected from the group consisting of monocytes, macrophages, B-cells, CD4⁺ cells, CD8⁺ cells, white blood cells, chondrocytes, endothelial cells, fibroblasts, breast endothelial cells and hematopoietic stem cells.

22. A method to detect disease in a patient comprising:
administration of HA to the patient sufficient to result in a transient increase in blood HA presence;
at lease one subsequent administration of HA to the patient sufficient to result in a transient increase in blood HA presence; and
identification and quantification of elimination kinetics of HA in the patient following the at least one subsequent administration of HA to the patient;
whereby alteration of the complexity of said elimination kinetics is indicative of the presence of the disease state.

23. The method of claim 22 wherein the disease is selected from the group consisting of cancer, arthritis and fibrosis.

24. A method to image cells or tissues of interest in a patient comprising:
administration of HA to the patient sufficient to result in a transient increase in HA presence proximal to the cell or tissue of interest;
at least one subsequent administration of HA to the patient sufficient to result in a transient increase in HA presence proximal to the cell or tissue of interest;
administration of an imaging dose of HA to the patient; and
imaging of the cell or tissue of interest for the imaging dose.

25. The method of claim 24 wherein the imaging dose of HA comprises HA complexed with a radionuclide.

26. The method of claim 25 wherein the radionuclide is selected from the group consisting of ²⁴¹Am, ¹³⁷Ba, ⁴⁷Ca, ¹³⁷Cs, ⁵¹Cr, ⁵⁷Co, ⁶⁰Co, ⁶⁴Cu, ¹⁸F, ⁶⁷Ga, ¹⁹⁸Au, ¹²³I, ¹²⁵I, ¹³¹I, ¹⁹²Ir, ⁵⁵Fe, ⁵⁹Fe, ⁸⁵Kr, ²¹⁰Pb, ¹⁹⁷Hg, ²⁰³Hg, ³²P, ⁴²K, ²²⁶Ra, ¹⁰⁵Ru, ⁷⁵Se, ⁸⁵Sr, ^{87m}Sr, ²⁴Na, ³⁵S, ^{99m}Tc, ²⁰¹Tl, ³H, ¹³³Xe and ¹⁶⁹Yb.

27. The method of claim 24 of whereby imaging of the cell or tissue of interest is performed using magnetic resonance.

28. The method of claim 27 whereby apparent diffusion coefficient imaging is performed.

29. The method of claim 27 or 28 whereby the imaging dose of HA contains magnetic resonance imaging marker.

30. The method of claim 29 wherein the magnetic resonance imaging marker is selected from the group comprising ¹³C, ¹³P, ¹¹B and ¹⁹F.
